(19) United States
(12) Patent Application Publication
(10) Pub. No.: US 2024/0409885 A1
(43) Pub. Date: Dec. 12, 2024

(54) NOVEL BACILLUS VELEZENSIS GYUN-1190 STRAIN AND USE THEREOF

(71) Applicant: Andong National University Industry-Academic Cooperation Foundation, Andong-si (KR)

(72) Inventors: Yong Ho JEON, Andong-si (KR); Hyeon Jin PARK, Gimcheon-si (KR)

(73) Assignee: Andong National University Industry-Academic Cooperation Foundation, Andong-si (KR)

(21) Appl. No.: 18/485,314

(22) Filed: Oct. 12, 2023

(30) Foreign Application Priority Data
Jun. 9, 2023 (KR) 10-2023-0074197

(51) Int. Cl. C12N 1/20; A01P 3/00

(52) U.S. Cl. CPC C12N 1/205; A01P 3/00; C12R 2001/07

(57) ABSTRACT

A novel Bacillus velezensis GYUN-1190 strain and uses thereof are proposed. Specifically, a plant disease control composition and method are proposed. The composition includes Bacillus velezensis GYUN-1190 strain (accession No: KACC 92512P), which is an antagonistic bacteria. The method includes a step of treating a plant part, soil, or a plant seed with the plant composition. The composition including the novel Bacillus velezensis GYUN-1190 strain, according to the present disclosure, exhibits excellent antagonistic activity against plant pathogens, thereby enabling cultivation of plants and fruits having high commercial values and highly safe. In addition, with the use of the novel GYUN-1190 strain, it is possible to alleviate problems such as environmental pollution due to conventional pesticides and human poisoning due to pesticide residues remaining in the body, thereby increasing the production of plants and fruits in a safer and eco-friendly way. Therefore, the present disclosure has a highly economically useful effect.

### STATEMENT REGARDING GOVERNMENT SPONSORED RESEARCH

This invention was supported by the Korea Institute of Planning and Evaluation for Technology in Food, Agriculture, and Forestry (IPET) funded by the Ministry of Agriculture, Food, and Rural Affairs (MAFRA) of Republic of Korea. [Research Program name: “Crop Virus and Pest Control Industrial Technology Development Program”; Research Project name: “Investigation of fungicide resistance mechanism of Colletotrichum spp. causing pepper anthracnose and development of control method”; Project Serial Number: 3200425; Research Project Number: 320042-5]

### CROSS REFERENCE TO RELATED APPLICATION

The present application claims priority to Korean Patent Application No. 10-2023-0074197, filed Jun. 9, 2023, the entire contents of which is incorporated herein for all purposes by this reference.

### REFERENCE TO AN ELECTRONIC SEQUENCE LISTING

The contents of the electronic sequence listing (PANY-119-Sequence_Listing.xml; Size: 3,837 bytes; and Date of Creation: Oct. 10, 2023) is herein incorporated by reference in its entirety.

### BACKGROUND OF THE INVENTION

### 1. Field of the Invention

The present disclosure relates to a novel Bacillus velezensis GYUN-1190 strain and a plant disease control agent using the strain.

### 2. Description of the Related Art

To control plant diseases, worldwide countries have been manufacturing and using organic compound-based pesticides. However, such existing pesticides have diverse problems such as environmental pollution, human poisoning by pesticide residue, and emergence of pesticide-resistant pathogens. Therefore, eco-friendly plant disease control (biological control) technology using microorganisms such as bacteria or fungi is gaining attention as an alternative to conventional plant disease control technology using organic compound-based pesticides.

Particularly, apple anthracnose and pepper anthracnose are diseases caused by a pathogenic fungus of the genus Colletotrichum and develop a light brown rounded pattern on the surface of fruits, resulting in the fruits decaying in the end. Apple anthracnose was first reported in England by Berkeley in 1856, and the causative organism of anthracnose, the genus Colletotrichum, is a pathogenic fungus distributed worldwide with a wide host range. Apple anthracnose in Korea mostly occurs from late July to late August and declines after the middle of September. Apple anthracnose is a deadly fruit disease which causes severe damage to orchardists because it also occurs a lot even during storage.

Currently, compound pesticides such as Dithianon, Carbendazium, Chlorothalonill, Azoxystrobin, and Mancozeb are used to control the anthracnose, but the existing pesticides cause many problems such as ecosystem destruction and inadequate control effect due to drug resistance.

Therefore, there is a need to develop a new biological control agent which is less toxic and does not pollute the environment.

### DOCUMENTS OF RELATED ART

### Patent Document

(Patent Document 0001) Korean Patent No. 10-0968680

### SUMMARY OF THE INVENTION

The inventors have identified a novel Bacillus velezensis GYUN-1190 strain which has excellent control effect on plant pathogens, and completed present disclosure by confirming the usability of the strain as a microbial product.

Therefore, the present disclosure provides an antagonistic bacteria Bacillus velezensis GYUN-1190 strain (accession No: KACC 92512P).

Further, the present disclosure provides a plant disease control composition including the Bacillus velezensis GYUN-1190 strain, a natural volatile compound produced from the strain, a culture of the strain, a condensate of the culture, a dry solid of the culture, or an extract of the culture, as an active ingredient.

Further, the present disclosure provides a method of controlling a plant disease, the method including treating a plant part, soil, or a plant seed with the plant disease control composition of the present disclosure.

Therefore, the present disclosure provides an antagonistic bacteria Bacillus velezensis GYUN-1190 strain (accession No: KACC 92512P).

Further, the present disclosure provides a plant disease control composition including the Bacillus velezensis GYUN-1190 strain of the present disclosure, a natural volatile compound produced from the strain, a culture of the strain, a condensate of the culture, a dry solid of the culture, or an extract of the culture, as an active ingredient.

In one embodiment of the present disclosure, the composition may have an activity of inhibiting spore germination and growth of a pathogenic fungus, Colletotrichum fructicola or Colletotrichum siamense.

In one embodiment of the present disclosure, the plant disease may be apple anthracnose.

Further, the present disclosure provides a plant disease control method including treating a plant part, soil, or a plant seed with the composition of the present disclosure.

In one embodiment of the present disclosure, the plant disease may be apple anthracnose.

In one embodiment of the present disclosure, the apple anthracnose may be caused by a pathogenic fungus, Colletotrichum fructicola or Colletotrichum siamense.

The plant disease control agent using the novel Bacillus velezensis GYUN-1190 strain according to the present disclosure has excellent antagonistic activity against plant pathogens, thereby enabling cultivation of plants and fruits having high commercial values and highly safe. Further, by using the novel GYUN-1190 strain, it is possible to alleviate problems such as environmental pollution due to conventional pesticides made of chemical compounds and human poisoning due to pesticide residues remaining in the body, and to increase the production of plants and fruits in a safer and eco-friendly way. Therefore, the present disclosure has a highly economically useful effect.

### BRIEF DESCRIPTION OF THE DRAWINGS

FIG. 1 shows an analysis result of a plant pathogen inhibitory activity of each of several strains which are isolated from soil and dual-cultured with, Colletotrichum fructicola GYUN-10893 fungus as a plant pathogen;

FIG. 2 shows an analysis result of the growth inhibition of the plant pathogen, Colletotrichum fructicola GYUN-10893 fungus, cultured using a sandwich culturing method to investigate the plant pathogen inhibitory activity of a volatile organic compound derived from each of several strains isolated from soil;

FIG. 3 shows an analysis result of the plant pathogen inhibitory activity of a culture filtrate of each of several strains isolated from soil;

FIG. 4 shows the spore germination inhibitory activity of a culture filtrate of a novel Bacillus velezensis GYUN-1190 strain against the plant pathogen, Colletotrichum fructicola GYUN-10893 fungus;

FIGS. 5A and 5B show the apple anthracnose disease control effect of the novel Bacillus velezensis GYUN-1190 strain, in which FIG. 5A shows a result of an experiment in which each of a cell suspension (S GYUN-1190) of the novel strain GYUN-1190 and a culture filtrate (CF GYUN-1190) of the novel strain GYUN-1190 was mixed with a spore suspension of the plant pathogen Colletotrichum fructicola GYUN-10893, each of the mixtures was cultured on a solid medium, a growth inhibitory effect on the pathogen on the culture medium was investigated, and FIG. 5B shows a result of an experiment in which each of the cultures of the respective mixtures was inoculated on a wound of an apple, and the apple anthracnose disease inhibitory effect was checked at predetermined time intervals; and

FIG. 6 shows a result of an experiment in which each of a cell suspension of the novel strain of the present disclosure and a culture filtrate of the novel strain was mixed with a spore suspension of the pathogenic fungus, Colletotrichum fructicola GYUN-10893, and each mixture was inoculated on a wounded apple to investigate a control effect on apple anthracnose over time.

### DESCRIPTION OF THE PREFERRED EMBODIMENTS

The present disclosure features providing a novel Bacillus velezensis GYUN-1190 strain which has an excellent control effect on plant pathogens.

The inventors of the present disclosure identified a new strain with excellent antifungal activity against plant pathogens isolated from soil, named it “Bacillus velezensis GYUN-1190” and deposited it to Korean Agricultural Culture Collection (KACC) on May 10, 2023 under an accession number of KACC 92512P.

The novel Bacillus velezensis GYUN-1190 strain has excellent antifungal activity against plant pathogens.

In one embodiment of the present disclosure, the growth of an apple anthracnose pathogen, Colletotrichum fructicola GYUN-10893 fungus, was effectively inhibited by dual culturing of the plant pathogenic fungus and the novel GYUN-1190 strain.

In another embodiment, an experiment was conducted to confirm whether the novel Bacillus velezensis GYUN-1190 strain of the present disclosure could produce a volatile organic compound and whether the volatile organic compound has antimicrobial activity against plant pathogens. A cultured medium of the novel GYUN-1190 strain and a cultured medium of plant pathogenic fungus were stacked like a sandwich form, and it was observed whether the volatile organic compound of the novel GYUN-1190 strain could inhibit mycelial growth of the pathogenic fungus. As a result, it was confirmed that the mycelial growth of the pathogenic fungus was effectively inhibited by the volatile organic compound.

The antimicrobial activity of the Bacillus velezensis GYUN-1190 strain against plant pathogens was also exhibited by volatile organic compounds produced by the new GYUN-10701 strain, and a culture product (culture filtrate) including secondary metabolites of the new GYUN-10701 strain.

Furthermore, the inventors confirmed that the isolated and identified novel GYUN-1190 strain had inhibitory activity against the apple anthracnose pathogen, Colletotrichum fructicola GYUN-10893 fungus, and investigated whether the novel strain has a control effect on apple anthracnose by inoculating a mixture of the novel GYUN-1190 strain and the apple anthracnose pathogen on a wounded apple. The inventors checked the degree of progress of anthracnose hourly and confirmed that in the group inoculated with the novel GYUN-1190 strain, the incidence rate of the apple anthracnose was significantly reduced, and that the anthracnose control effect of the novel GYUN-1190 strain was better than Tebuconazole and Pyraclostrobin, which are chemical fungicides.

Therefore, the present disclosure provides a plant disease control composition including Bacillus velezensis GYUN-1190 strain, a natural volatile compound produced from the strain, a culture of the strain, a condensate of the culture, a dry solid of the culture, or an extract of the culture, as an active ingredient.

In particular, the composition of the present disclosure has an effect of inhibiting both spore germination and growth of Colletotrichum fructicola or a Colletotrichum siamense, which are pathogenic fungi.

Therefore, the composition of the present disclosure can control the apple anthracnose caused by the pathogenic fungus, Colletotrichum fructicola or Colletotrichum siamense.

In addition, the composition of the present disclosure can use all of the novel GYUN-1190 strain, a natural volatile compound produced from the strain, a culture of the strain, a condensate of the culture, a dry solid of the culture, and an extract of the culture as an active ingredient. Here, the culture may be a culture (cell suspension) including the novel GYUN-1190 strain or a culture solution (culture filtrate) including metabolites secreted by the novel GYUN-1190 strain.

Furthermore, the present disclosure provides a plant disease control method including a step of treating a plant part, soil, or a plant seed with the composition of the present disclosure.

The method of the present disclosure can be performed by treating a plant part, soil, or a plant seed with the novel GYUN-1190 strain, a natural volatile compound produced from the strain, a culture of the strain, a condensate of the culture, a dry solid of the culture, or an extract of the culture. For the treatment, either or both of foliar application and drenching may be performed.

Hereinafter, exemplary embodiment of the present disclosure will be described in detail. These exemplary embodiments are presented to describe the present disclosure in greater detail and thus are not intended to limit the scope of the present disclosure.

### Example 1

Isolation and Identification of Novel Bacillus velezensis GYUN-1190 Strain

Soil samples were collected from an apple field in Jecheon, Chungcheongbuk-do, Korea. Next, to select a new antagonistic bacterium, the soil was suspended in sterilized SDW, and the suspension was diluted, and a culture medium was streaked with the diluted suspension. Specifically, the soil 5 g and sterile water 40 ml were put in a conical tube and shaken for 1 minute using a vortex mixer. The sample was serially diluted seven times and 100 μL of the sample was dispensed to a TSA plate. A tryptic soy agar (TSA) containing 15 g of a tryptic soy broth and 8 g of agar was used as a culture medium. Multiple microbial colonies were then collected, and each fungal colony was tested to confirm whether it exhibited an antibacterial activity against an apple anthracnose pathogen.

### <1-1> Selection of Strain Having Antimicrobial Activity Against Apple Anthracnose Pathogen Through Dual-Culture

To this end, analysis of antimicrobial activity of a cultured strain of each fungal colony against Colletotrichum fructicola GYUN-10893, which is an apple anthracnose pathogen, was performed on a PDK flat medium through dual-culturing performed in a 25° C. incubator.

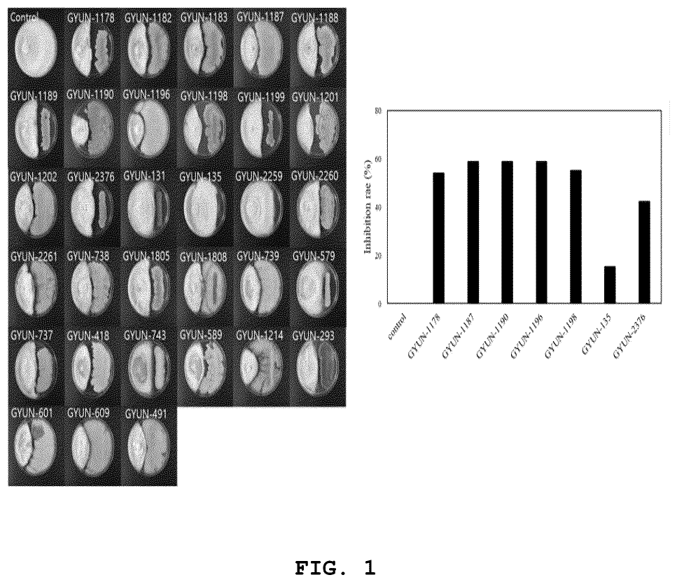

As a result, as shown in FIG. 1, six strains with excellent antimicrobial activity against the apple anthracnose pathogen were selected from the tested fungal colonies, and especially GYUN-1187, GYUN-1190, and GYUN-1196 strains showed superior antimicrobial activity to the other strains.

### <1-2> Analysis of Plant Pathogen Inhibitory Activity of Volatile Organic Compounds of Selected Strain

Volatile organic compounds produced from the selected strains were analyzed for plant pathogen inhibitory activity. To this end, a sandwich culture method was used for the investigation. Specifically, GYUN-10893 plug, which is an apple anthracnose pathogen, was placed in the center of a PDA medium, and TSA media were streaked with the respective strains selected in Step <1-1>, and the sandwich culturing was performed in a 28° C. incubator for 14 days. Here, the control group was a non-inoculated TSA medium.

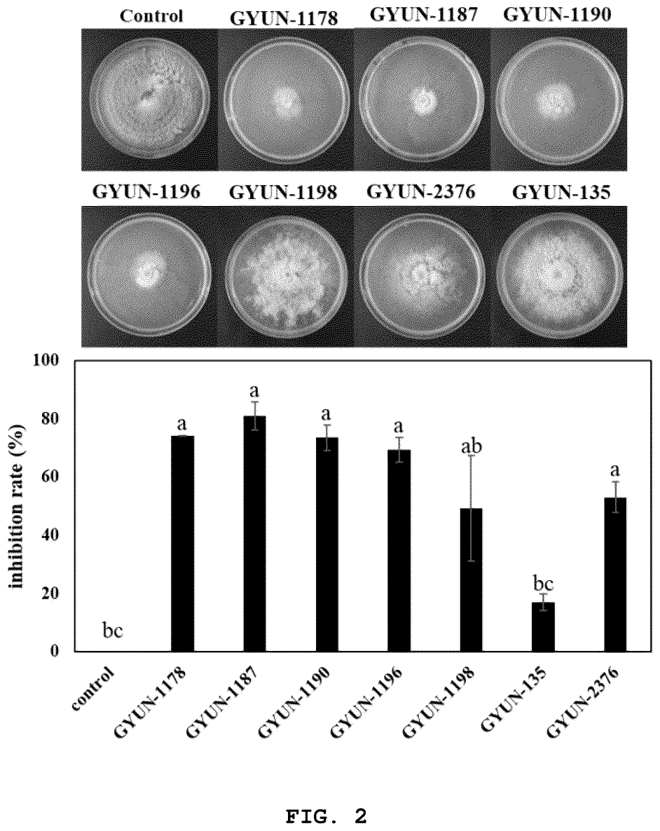

As a result, as shown in FIG. 2, the volatile organic compounds produced from the GYUN-1178, GYUN-1187, GYUN-1190 and GYUN-1196 strains showed superior antimicrobial activity against the plant pathogen, Colletotrichum fructicola GYUN-10893, to the other strains.

### <1-3> Analysis of Antagonism of Secondary Metabolites of the Selected Strain

To analyze antagonistic activity of secondary metabolites of each of the strains used for analysis in <1-2>, the culture filtrates of each strain were obtained and dual-cultured with the Colletotrichum fructicola GYUN-10893 pathogen. The culture filtrates were obtained through the steps of: culturing each of the strains on a TSB liquid medium overnight; inoculating a 1% culture solution of each strain on 100 ml of the TSB liquid medium; culturing in a 28° C. incubator with shaking at 180 rpm for 3 days; and filtering through a syringe filter using a syringe. In addition, for the dual-culture, a PDK medium was streaked with 100 ul of a GYUN-10893 pathogen spore suspension (10⁵ conidia/ml), and 10 ul of the culture filtrate for each strain was dropped onto a paper disk and cultured in a 25° C. incubator for 2 days.

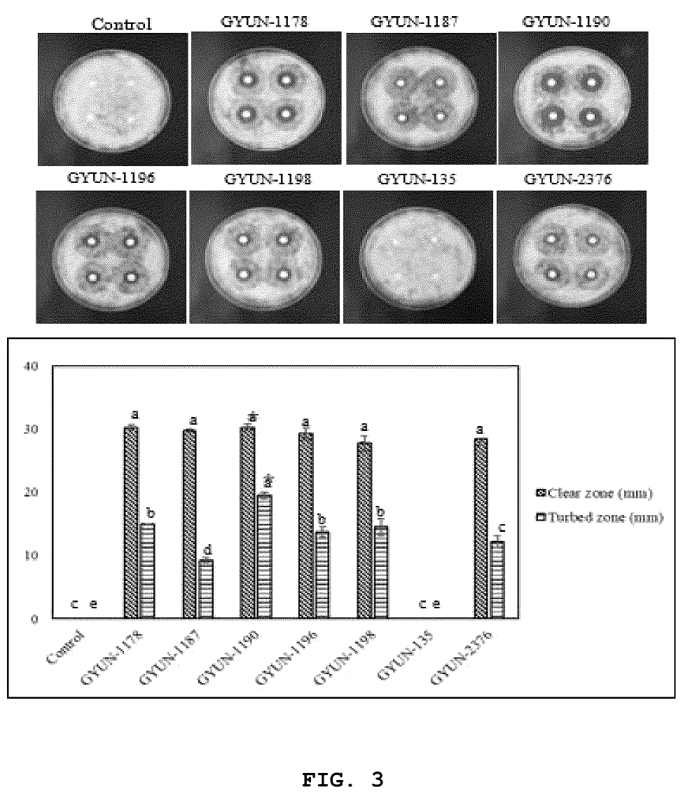

As a result, as shown in FIG. 3, among the selected strains, the GYUN-1190 strain was found to have the most excellent antimicrobial effect compared to the other strains. The analysis results revealed that a clear zone and a turbid zone had sizes of 30.0 mm and 19.5 mm, respectively.

<1-4> Identification of Novel Bacillus velezensis GYUN-1190 Strain

For the molecular biological identification of the GYUN-1190 strain finally selected in <1-3>, PCR amplification was performed on 16S rDNA of the isolated microorganism. The genomic DNA was extracted using Biofact Genomic DNA prep kit (Biofact co, Korea) according to the manufacturer's test manual. The strain isolated and cultured for extraction was cultured in a PDA medium at 25° C. for 7 days. The spores and hyphae formed on the medium were transferred to a mortar using a sterilized scalpel, liquid nitrogen was added thereto, and the content in the mortar was ground with a pestle. The finely ground powder was transferred to a 1.5-ml tube, 200 μl of GD1 and 5 μl of Proteinase K (20 mg/ml) were added to the powder and mixed for 1 minute using a vortex mixer, and the mortar was retained in a 65° C. water bath for 10 minutes. Next, 200 ul of GD2 was added to the mortar, and the contents in the mortar was mixed for 10 seconds using a vortex mixer and centrifuged at 13,000 rpm for 5 minutes. After the centrifugation, the supernatant thereof was transferred to a new 1.5-ml tube, 200 ul of GB was added thereto, and the contents was inverted 10 times. A spin column was loaded in a 2-ml collection tube, and 200 μl of Help B was added thereto, followed by centrifugation at 10,000 rpm for 30 seconds. The liquid resulting from the centrifugation was discarded, and the solution having undergone the inverting was added to the remainder obtained through the centrifugation. The mixture was centrifuged at 7,000 rpm for 1 minute, a spin column and a collection tube were installed. 500 μl of WB was added, followed by centrifugation at 13,000 rpm for 30 seconds, and the filtrate was discarded. The process (i.e., sequential steps of the addition of WB, centrifugation, and the removal of the filtrate) was performed twice. No-load rotation (i.e., idle operation) was performed at 13,000 rpm for 3 minutes. After the no-load rotation, a spin column was loaded in a new 1.5-ml tube, 30 ul of a DNA hydration solution was poured into the spin column, left at room temperature for 2 minutes, centrifuged at 13,000 rpm for 2 minutes twice, and genomic DNA was collected. The remaining DNA after the experiment was stored in a refrigerator at −80° C. PCR analysis of the 16S rDNA region was performed using the genomic DNA extracted for strain analysis. To amplify the 16S rDNA region, Primer 27F (5′-AGAGTTTGATCATGGGTCAG-3′) and 1492R Primer (5′-GGATACCTTGTTACGACTT-3′ ITS4) were used. The PCR reaction was as follows: initial denaturation at 95° C. for 1 minute, denaturation at 95° C. for 20 seconds, annealing at 52° C. for 40 seconds, 35 cycles of extension at 72° C. for 1 minute, extension at 72° C. for 5 minutes, and preservation at 4° C. The resultant was used as a sample for ITS gene sequencing. The obtained DNA sequence was compared with existing DNA sequences through the BLAST search of the National Center for Biotechnology Information (NCBI), and the DNA sequence for the 16S rDNA region was confirmed as represented by SEQ ID NO: 1.

As a result, the isolated rhizosphere soil fungi in the present disclosure were found to belong to the genus Bacillus velezensis and was confirmed to be a new strain which have not existed. The inventors of the present disclosure named it “Bacillus velezensis GYUN-1190”, and deposited it to Korean Agricultural Culture Collection (KACC), Agricultural Microbiology Division, National Institute of Agricultural Sciences under an accession number of KACC 92512P.

### Example 2

### Analysis of Plant Pathogen Inhibitory Activity of Novel Bacillus Velezensis GYUN-1190 Strain

The inventors of the present disclosure selected Bacillus velezensis GYUN-1190 strain that exhibited the highest antimicrobial activity against especially plant pathogens through the above-described experiment among the strains isolated from soil and conducted another experiment to confirm an antimicrobial activity of the novel Bacillus velezensis GYUN-1190 strain against plant pathogens.

The Bacillus velezensis GYUN-1190 strain culture solution (culture filtrate) obtained as in Example 1 and a spore suspension (1×10⁵ conidia/ml) of GYUN-10893 strain, which is a plant pathogen, were mixed in a ratio of 1:1, the mixture was dropped by 20 ul onto each slide glass, and the strains were cultured in a 25° C. incubator. During the culturing, the spore inhibition rate of the plant pathogen was observed at predetermined time intervals (0 h, 6 h, 12 h, and 24 h) through microscopy. An untreated TSB medium was used as a negative control group, and Bacillus velezensis GYUN-135 was used as a positive control group.

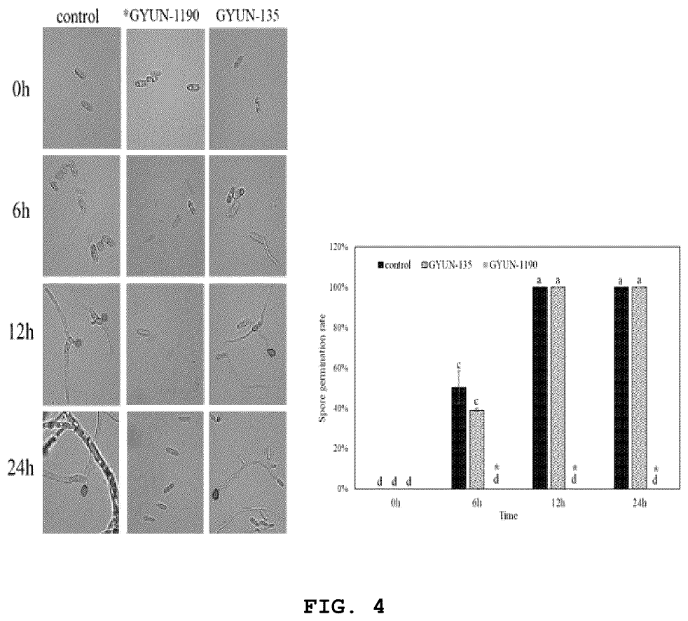

As a result, as shown in FIG. 4, the group treated with the culture filtrate of Bacillus velezensis GYUN-1190 strain showed 100% inhibition of spore germination of the plant pathogen.

### Example 3

Verification of Apple Anthracnose Control Effect of Novel Bacillus velezensis GYUN-1190 Strain

The plant pathogen inhibitory activity of the novel Bacillus velezensis GYUN-1190 strain was analyzed on using a solid medium. To this end, a cell suspension and culture filtrate of the novel GYUN-1190 strain were mixed with a spore suspension (concentration: 1×10³, 1×10⁵/ml) of the GYUN-10893 strain (apple anthracnose pathogen), and the mixture was cultured for 0, 8, 24, 48 hours. Afterwards, a streptomycin-added PDA medium was streaked with the culture at predetermined timing. To prepare a positive control group, the apple anthracnose pathogen was treated with Tebuconazole and Pyraclostrobin, which are chemical fungicides. To prepare a negative control group, the apple anthracnose pathogen was treated with sterile distilled water (SDW). Each test group was cultured in a 25° C. incubator, and the inhibition rate of each test group was determined by counting the number of fungal colonies.

In addition, four wounds were made with a sterile needle for each apple, and 10 ul of a mixed strain solution that was prepared in the same manner as in the case of the solid medium was inoculated on each wounded apple fruit at predetermined time intervals. The experiment was repeated three times, and a 25° C. incubator was used for the culturing in each experiment.

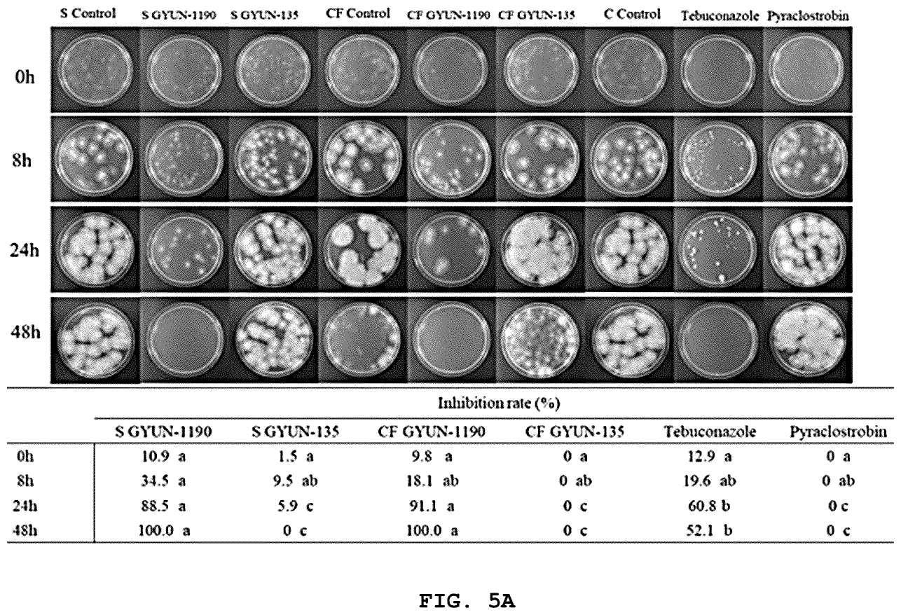

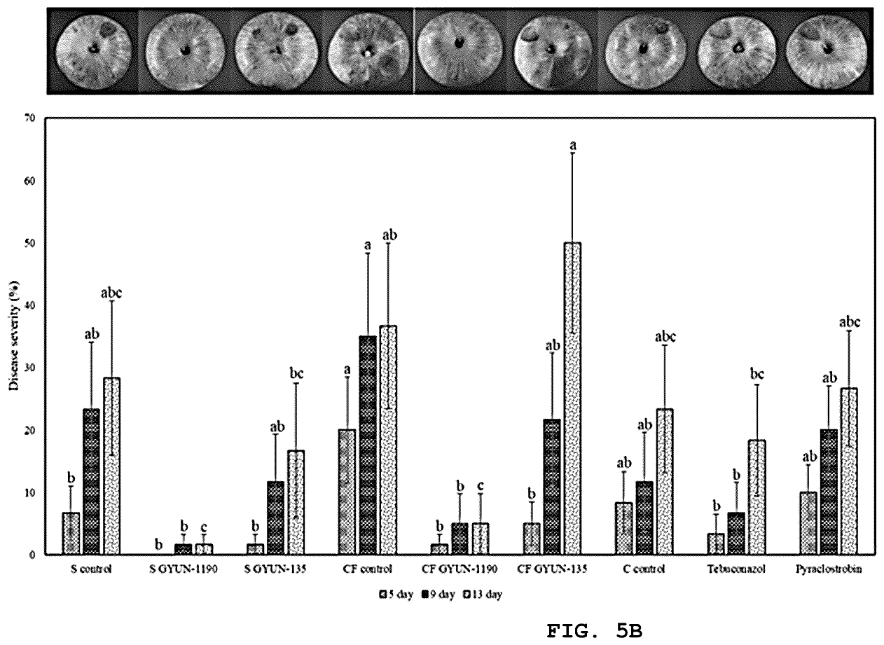

As a result, as shown in FIGS. 5A and 5B, in the groups treated with the cell suspension(S) and culture solution (CF) of the Bacillus velezensis GYUN-1190 strain of the present disclosure, the growth of the apple anthracnose pathogen, GYUN-10893 strain, was effectively inhibited. The results of 24-hour culturing confirmed that the groups treated with the culture filtrate of the GYUN-1190 strain 91.1% reduction in a growth rate, the positive control group treated with Tebuconazole exhibited 60.8% reduction in a growth rate, and the group treated with Pyraclostrobin exhibited no reduced in a growth rate (see FIG. 5A).

In addition, the results of confirming the inhibitory effect on the plant pathogen in an apple confirmed that when compared with the negative control group and the positive control group, the cell suspension and culture filtrate of the GYUN-1190 strain exhibited an inhibitory effect on the development of apple anthracnose by 94.12% and 86.36%, respectively (see FIG. 5B).

In another experiment, the inventors of the present disclosure mixed a spore suspension (concentration: 1×10⁵ cells/mL) of the apple anthracnose pathogen, GYUN-1089, with and each of the strains used in the above-described experiment, 10 μL of each mixture was inoculated on wounded apples, and the increase in the area of each wound was observed at predetermined time intervals (0 h, 8 h, 24 h, and 48 h).

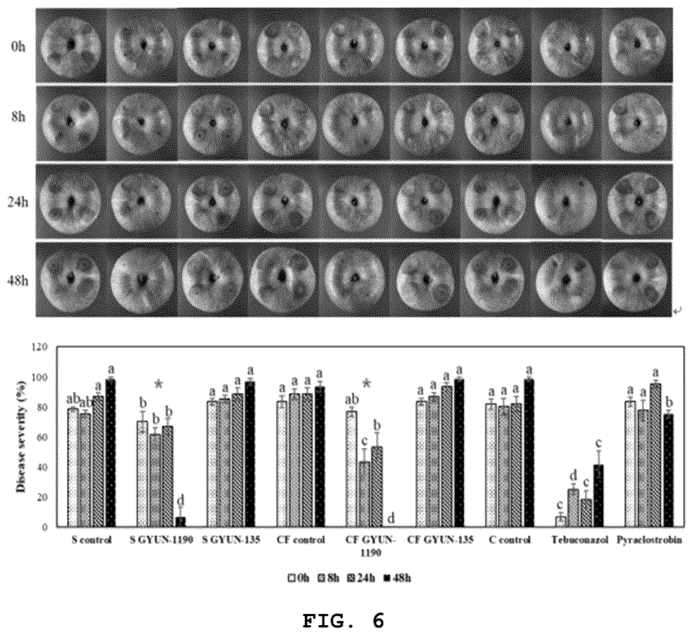

As a result, as shown in FIG. 6, the positive control group which was treated with Tebuconazole inhibited a significantly high inhibitory effect on the development of apple anthracnose at an early stage, but after 48 hours, exhibited a lower inhibitory effect than the group treated with the cell suspension or culture filtrate of the GYUN-1190 strain of the present disclosure. Specifically, after 48 hours, in the group treated with the cell suspension of the GYUN-1190 strain, the development of apple anthracnose was inhibited by 93.22%, and in the group treated with the culture filtrate of the GYUN-1190 strain, the development of apple anthracnose was inhibited by nearly 100%.

From the above results, the inventors of the present disclosure confirmed that the novel Bacillus velezensis GYUN-1190 strain has excellent antimicrobial activity against plant pathogens and can be used as a plant disease control agent.

The present disclosure has been described through preferred embodiments and examples, but the ordinarily skilled in the art would appreciate that the embodiments and examples can be implemented in many different forms without departing from the essential features of the present disclosure. Therefore, the disclosed embodiments and examples should be considered not from a restrictive perspective but from an illustrative perspective. The scope of present disclosure is not defined by the above description but by the appended claims, and all modifications, variations, and equivalents thereto will be construed as falling within the scope of the present disclosure.

### ACCESSION NUMBER

Depository authority: Korean Agricultural Culture Collection (KACC), Agricultural Microbiology Division, National Institute of Agricultural Sciences Accession number: KACC92512P
Accession date: 20230510

### SEQUENCE LIST

Attachment of electronic file of Sequence list

What is claimed is:

1. An antagonistic bacterial strain named Bacillus velezensis GYUN-1190 (accession No: KACC 92512P).

2. A composition for controlling a plant disease, the composition comprising, as an active ingredient, the strain of claim 1, a natural volatile compound produced from the strain, a culture of the strain, a condensate of the culture, a dry solid of the culture, or an extract of the culture.

3. The composition of claim 2, wherein composition has an activity of inhibiting spore germination and growth of, a pathogenic fungus, Colletotrichum fructicola or Colletotrichum siamense.

4. The composition of claim 2, wherein the plant disease is apple anthracnose.

5. A plant disease control method comprising treating a plant part, soil, or a plant seed with the composition of claim 2.

6. The method of claim 5, wherein the plant disease is apple anthracnose.

7. The method of claim 6, wherein the apple anthracnose is caused by a pathogenic fungus, Colletotrichum fructicola or Colletotrichum siamense.


SEQUENCE LISTING (1 sequence)

SEQ ID NO: 1 - DNA, 1429 bp, synthetic construct
  misc_feature 1..1429: note = GYUN-1190 16S rDNA sequence
  source 1..1429: mol_type = other DNA
ttcggcggctggctccataaaggttacctcaccgacttcgggtgttacaaactctcgtggtgtgacgggcggtgtgtacaaggcccgggaacgtattcaccgcggcatgctgatccgcgattactagcgattccagcttcacgcagtcgagttgcagactgcgatccgaactgagaacagatttgtgggattggcttaacctcgcggtttcgctgccctttgttctgtccattgtagcacgtgtgtagcccaggtcataaggggcatgatgatttgacgtcatccccaccttcctccggtttgtcaccggcagtcaccttagagtgcccaactgaatgctggcaactaagatcaagggttgcgctcgttgcgggacttaacccaacatctcacgacacgagctgacgacaaccatgcaccacctgtcactctgcccccgaaggggacgtcctatctctaggattgtcagaggatgtcaagacctggtaaggttcttcgcgttgcttcgaattaaaccacatgctccaccgcttgtgcgggcccccgtcaattcctttgagtttcagtcttgcgaccgtactccccaggcggagtgcttaatgcgttagctgcagcactaaggggcggaaaccccctaacacttagcactcatcgtttacggcgtggactaccagggtatctaatcctgttcgctccccacgctttcgctcctcagcgtcagttacagaccagagagtcgccttcgccactggtgttcctccacatctctacgcatttcaccgctacacgtggaattccactctcctcttctgcactcaagttccccagtttccaatgaccctccccggttgagccgggggctttcacatcagacttaagaaaccgcctgcgagccctttacgcccaataattccggacaacgcttgccacctacgtattaccgcggctgctggcacgtagttagccgtggctttctggttaggtaccgtcaaggtgccgccctatttgaacggcacttgttcttccctaacaacagagctttacgatccgaaaaccttcatcactcacgcggcgttgctccgtcagactttcgtccattgcggaagattccctactgctgcctcccgtaggagtctgggccgtgtctcagtcccagtgtggccgatcaccctctcaggtcggctacgcatcgtcgccttggtgagccgttacctcaccaactagctaatgcgccgcgggtccatctgtaagtggtagccgaagccaccttttatgtctgaaccatgcggttcaracaaccatccggtattagccccggtttcccggagttatcccagtcttacaggcaggttacccacgtgttactcacccgtccgccgctaacatcagggagcaagctcccatctgtccgctcgactgcatgtatagc